(19) United States
(12) Patent Application Publication
(10) Pub. No.: US 2015/0342672 A1
(43) Pub. Date: Dec. 3, 2015

(54) DOUBLE MICRO-ELECTRODE CATHETER

(71) Applicant: BOSTON SCIENTIFIC SCIMED, INC., MAPLE GROVE, MN (US)

(72) Inventors: ROBERT F. BENCINI, SUNNYVALE, CA (US); JOSEF V. KOBLISH, SUNNYVALE, CA (US)

(73) Assignee: BOSTON SCIENTIFIC SCIMED, INC., MAPLE GROVE, MN (US)

(21) Appl. No.: 14/716,405

(22) Filed: May 19, 2015

Related U.S. Application Data
(60) Provisional application No. 62/005,551, filed on May 30, 2014.

(51) Int. Cl. A61B 18/14; A61B 5/00; A61B 8/12; A61B 5/042; A61B 5/01

(52) U.S. Cl. CPC A61B 18/1492; A61B 5/0422; A61B 5/01; A61B 8/12; A61B 5/6852; A61B 2018/00577

(57) ABSTRACT

Medical devices and methods for making and using medical devices are disclosed. An example medical device may include a catheter for use in cardiac mapping and/or ablation. The catheter may include an elongate catheter shaft having a distal ablation electrode region capable of ablating tissue. A plurality of micro-electrode assemblies may be coupled to the distal ablation electrode region. At least one of the micro-electrode assemblies may include an inner electrode and an outer electrode disposed at least partially around the inner electrode. At least one of the inner electrode and the outer electrode may comprise a sensor.

### CROSS-REFERENCE TO RELATED APPLICATIONS

This application claims priority under 35 U.S.C. §119 to U.S. Provisional Application Ser. No. 62/005,551, filed May 30, 2014, the entirety of which is incorporated herein by reference.

### TECHNICAL FIELD

The present disclosure pertains to medical devices, and methods for manufacturing medical devices. More particularly, the present disclosure pertains to for cardiac mapping and/or ablation.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a catheter for use in cardiac mapping and/or ablation. The catheter may include an elongate catheter shaft having a distal ablation electrode region capable of ablating tissue. A plurality of micro-electrode assemblies may be coupled to the distal ablation electrode region. At least one of the micro-electrode assemblies may include an inner electrode and an outer electrode disposed at least partially around the inner electrode. At least one of the inner electrode and the outer electrode may comprise a sensor.

Alternatively or additionally to any of the embodiments above, the distal ablation electrode region includes a platinum ablation tip electrode.

Alternatively or additionally to any of the embodiments above, the distal ablation electrode region is rotatable relative to the catheter shaft.

Alternatively or additionally to any of the embodiments above, three or more micro-electrode assemblies are disposed along the distal ablation electrode region.

Alternatively or additionally to any of the embodiments above, the micro-electrode assemblies are spaced substantially equidistant from one another about the circumference of the distal ablation electrode region.

Alternatively or additionally to any of the embodiments above, only one of the inner electrode and the outer electrode comprises a sensor.

Alternatively or additionally to any of the embodiments above, both the inner electrode and the outer electrode comprises a sensor.

Alternatively or additionally to any of the embodiments above, the sensor includes a voltage sensor.

Alternatively or additionally to any of the embodiments above, the sensor includes a temperature sensor.

Alternatively or additionally to any of the embodiments above, the sensor includes an ultrasound sensor.

Alternatively or additionally to any of the embodiments above, the sensor includes a force sensor.

Alternatively or additionally to any of the embodiments above, the sensor includes a pressure sensor.

Alternatively or additionally to any of the embodiments above, the sensor includes an impedance sensor.

Alternatively or additionally to any of the embodiments above, the sensor includes an EGM sensor.

Another example catheter for use in cardiac mapping and/or ablation is disclosed. The catheter includes an elongate catheter shaft having a distal ablation tip electrode. A plurality of micro-electrode assemblies are coupled to the distal ablation tip electrode. At least one of the micro-electrode assemblies includes a first sensor, a second sensor, and a layer of insulation disposed between the first sensor and the second sensor. The first sensor, the second senor, or both includes a temperature sensor, an ultrasound sensor, a force sensor, a pressure sensor, an impedance sensor, or an EGM sensor.

Alternatively or additionally to any of the embodiments above, the first sensor and the second sensor are substantially congruent or geometrically similar.

Alternatively or additionally to any of the embodiments above, the first sensor and the second sensor have dissimilar shapes.

Alternatively or additionally to any of the embodiments above, the distal ablation tip electrode is rotatable relative to the catheter shaft.

Alternatively or additionally to any of the embodiments above, the distal ablation tip electrode includes three or more micro-electrode assemblies and wherein the micro-electrode assemblies are spaced substantially equidistant from one another about the circumference of the distal ablation tip electrode.

An example method for mapping and/or ablation cardiac tissue is disclosed. The method includes advancing a mapping and/or ablation catheter through a blood vessel to a position within a cardiac chamber. The catheter comprises an elongate catheter shaft having a distal ablation electrode region capable of ablating tissue and a plurality of micro-electrode assemblies coupled to the distal ablation electrode region. At least one of the micro-electrode assemblies includes an inner electrode and an outer electrode disposed at least partially around the inner electrode. At least one of the inner electrode and the outer electrode comprises a sensor. The method also includes activating the inner electrode, the outer electrode, or both.

Another example catheter for use in cardiac mapping and/or ablation may include an elongate catheter shaft having a distal ablation tip electrode. A plurality of micro-electrode assemblies may be coupled to the distal ablation tip electrode. At least one of the micro-electrode assemblies may include a first sensor, a second sensor, and a layer of insulation disposed between the first sensor and the second sensor. The first sensor, the second senor, or both may include a temperature sensor, an ultrasound sensor, a force sensor, a pressure sensor, an impedance sensor, or an EGM sensor.

Another example method for mapping and/or ablating cardiac tissue may include advancing a catheter through a blood vessel to a position within a cardiac chamber. The catheter may include an elongate catheter shaft having a distal ablation electrode region capable of ablating tissue. A plurality of micro-electrode assemblies may be coupled to the distal ablation electrode region. At least one of the micro-electrode assemblies may include an inner electrode and an outer electrode disposed at least partially around the inner electrode. At least one of the inner electrode and the outer electrode may comprise a sensor. The method may also include activating the inner electrode, the outer electrode, or both.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a plan view of an example cardiac mapping and/or ablation system;

FIG. 2 is a side view of a portion of an example cardiac mapping and/or ablation system;

FIG. 3 schematically illustrates an example micro-electrode assembly;

FIG. 4 schematically illustrates an example system including a plurality of micro-electrode assemblies;

FIG. 5 is a side view of a portion of an example cardiac mapping and/or ablation system;

FIG. 6 is a side view of a portion of an example cardiac mapping and/or ablation system;

FIG. 7 is a side view of a portion of an example cardiac mapping and/or ablation system;

FIG. 8 is a side view of a portion of an example cardiac mapping and/or ablation system;

FIG. 9 is a side view of a portion of an example cardiac mapping and/or ablation system;

FIG. 10 is a side view of a portion of an example cardiac mapping and/or ablation system;

FIG. 11 is a side view of a portion of an example cardiac mapping and/or ablation system;

FIG. 12 is a side view of a portion of an example cardiac mapping and/or ablation system;

FIG. 13 is a side view of a portion of an example cardiac mapping and/or ablation system;

FIG. 14 is a side view of a portion of an example cardiac mapping and/or ablation system;

FIG. 15 is a side view of a portion of an example cardiac mapping and/or ablation system;

FIG. 16 is a side view of a portion of an example cardiac mapping and/or ablation system;

FIG. 17 is a side view of a portion of an example cardiac mapping and/or ablation system;

FIG. 18 is a partial cross-sectional side view of an example cardiac mapping and/or ablation system;

FIG. 19 is a plan view of an example cardiac mapping and/or ablation system in a first configuration; and

FIG. 20 is a plan view of an example cardiac mapping and/or ablation system in a second configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

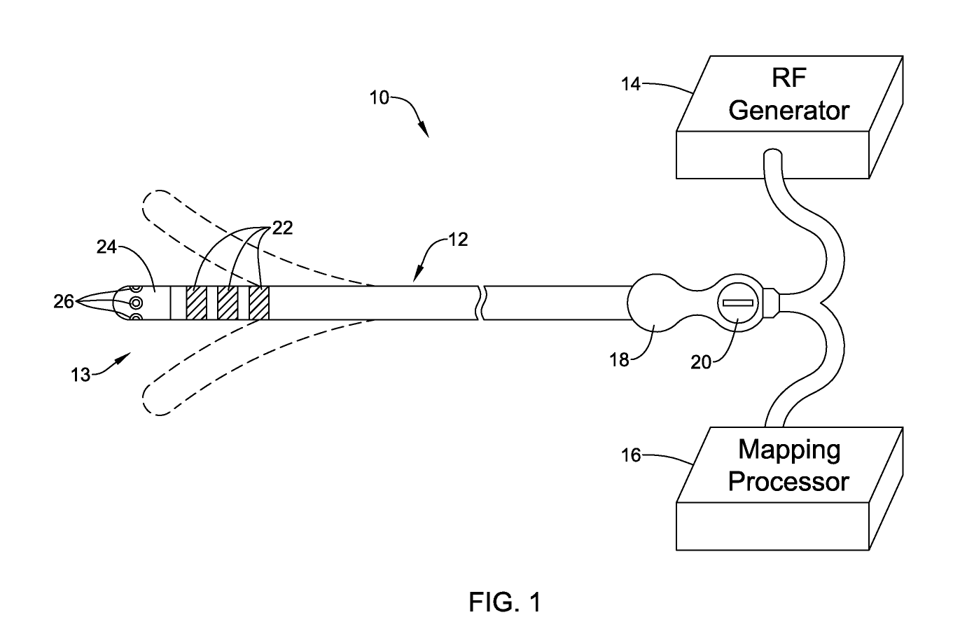

FIG. 1 illustrates an example cardiac mapping and/or ablation system 10. As shown in FIG. 1, system 10 may include an elongated member or catheter shaft 12, an RF generator 14, and a processor 16 (e.g., a mapping processor, ablation processor, and/or other processor). Illustratively, shaft 12 may be operatively coupled to at least one or more (e.g., one or both) of RF generator 14 and processor 16. Alternatively, or in addition, a device, other than shaft 12, that may be utilized to apply ablation energy to and/or map a target area, may be operatively coupled to at least one or more of RF generator 14 and processor 16. RF generator 14 may be capable of delivering and/or may be configured to deliver ablation energy to shaft 12 in a controlled manner in order to ablate target area sites identified by processor 16. Although the processor 16 and RF generator 14 may be shown as discrete components, these components or features of components may be incorporated into a single device. System 10 may include any of one or more other features, as desired.

In at least some embodiments, shaft 12 may include a handle 18, which may have an actuator 20 (e.g., a control knob or other actuator). The handle 18 (e.g., a proximal handle) may be positioned at a proximal end of shaft 12, for example. Illustratively, shaft 12 may include a flexible body having a having a distal portion which may include the one or more electrodes. For example, the distal portion of shaft 12 may include one or more of a plurality of ring electrodes 22, a distal ablation tip electrode 24, and a plurality of micro-electrodes or micro-electrode assemblies 26 disposed or otherwise positioned within and/or electrically isolated from distal ablation tip electrode 24.

Shaft 12 may be steerable to facilitate navigating the vasculature of a patient or navigating other lumens. Illustratively, a distal portion 13 of shaft 12 may be deflected by manipulation of actuator 20 to effect steering shaft 12. In some instances, distal portion 13 of shaft 12 may be deflected to position distal ablation tip electrode 24 and/or micro-electrode assemblies 26 adjacent target tissue or to position the distal portion 13 of shaft 12 for another suitable purpose. Additionally, or alternatively, distal portion 13 of shaft 12 may have a pre-formed shape adapted to facilitate positioning distal ablation tip electrode 24 and/or micro-electrode assemblies 26 adjacent a target tissue. Illustratively, the preformed shape of distal portion 13 of shaft 12 may be a radiused shape (e.g., a generally circular shape or a generally semi-circular shape) and/or may be oriented in a plane transverse to a general longitudinal direction of shaft 12. These are just examples.

In some instances, system 10 may be utilized in ablation procedures on a patient. Illustratively, shaft 12 may be configured to be introduced into or through vasculature of a patient and/or into or through any other lumen or cavity. In one example, shaft 12 may be inserted through the vasculature of the patient and into one or more chambers of the patient's heart (e.g., a target area). When in the patient's vasculature or heart, shaft 12 may be used to map and/or ablate myocardial tissue using the ring electrodes 22, micro-electrode assemblies 26, and/or distal ablation tip electrode 24. In some instances, distal ablation tip electrode 24 may be configured to apply ablation energy to myocardial tissue of the heart of a patient.

In some instances, micro-electrode assemblies 26 may be circumferentially distributed about a distal ablation tip electrode 24. Micro-electrode assemblies 26 may be capable of operating, or configured to operate, in unipolar or bipolar sensing modes. In some cases, micro-electrode assemblies 26 may define and/or at least partially form one or more bipolar microelectrode pairs. In an illustrative instance, shaft 12 may have three micro-electrode assemblies 26 distributed about the circumference of distal ablation tip electrode 24, such that the circumferentially spaced microelectrodes may form respective bipolar microelectrode pairs. Each bipolar microelectrode pair may be capable of generating, or may be configured to generate, an output signal corresponding to a sensed electrical activity (e.g., an electrogram (EGM) reading) of the myocardial tissue proximate thereto. Additionally or alternatively to the circumferentially spaced micro-electrode assemblies 26, shaft 12 may include one or more forward facing micro-electrode assemblies 26 (not shown). The forward facing micro-electrode assemblies 26 may be generally centrally located within distal ablation tip electrode 24 and/or at an end of a tip of shaft 12.

In some examples, micro-electrode assemblies 26 may be operatively coupled to processor 16 and the generated output signals from micro-electrode assemblies 26 may be sent to the processor 16 of ablation system 10 for processing in one or more manners discussed herein and/or for processing in other manners. Illustratively, an EGM reading or signal of an output signal from a bipolar microelectrode pair may at least partially form the basis of a contact assessment, ablation area assessment (e.g., tissue viability assessment), and/or an ablation progress assessment (e.g., a lesion formation/maturation analysis), as discussed below.

Distal ablation tip electrode 24 may be a suitable length and may have a suitable number of micro-electrode assemblies 26 positioned therein and spaced circumferentially and/or longitudinally about distal ablation tip electrode 24. In some instances, distal ablation tip electrode 24 may have a length of between one (1) mm and twenty (20) mm, three (3) mm and seventeen (17) mm, or six (6) mm and fourteen (14) mm. In one illustrative example, distal ablation tip electrode 24 may have an axial length of about eight (8) mm. Distal ablation tip electrode 24 may be formed from other otherwise include platinum and/or other suitable materials. These are just examples.

Processor 16 may be capable of processing or may be configured to process the electrical signals of the output signals from micro-electrode assemblies 26 and/or ring electrodes 22. Based, at least in part, on the processed output signals from micro-electrode assemblies 26 and/or ring electrodes 22, processor 16 may generate an output to a display (not shown) for use by a physician or other user. In instances where an output is generated to a display and/or other instances, processor 16 may be operatively coupled to or otherwise in communication with the display. Illustratively, the display may include various static and/or dynamic information related to the use of system 10. In one example, the display may include one or more of an image of the target area, an image of shaft 12, and information related to EGMs, which may be analyzed by the user and/or by a processor of system 10 to determine the existence and/or location of arrhythmia substrates within the heart, to determine the location of shaft 12 within the heart, and/or to make other determinations relating to use of shaft 12 and/or other elongated members.

System 10 may include an indicator in communication with processor 16. The indicator may be capable of providing an indication related to a feature of the output signals received from one or more of the electrodes of shaft 12. In one example of an indicator, an indication to the clinician about a characteristic of shaft 12 and/or the myocardial tissue interacted with and/or being mapped may be provided on the display. In some cases, the indicator may provide a visual and/or audible indication to provide information concerning the characteristic of shaft 12 and/or the myocardial tissue interacted with and/or being mapped.

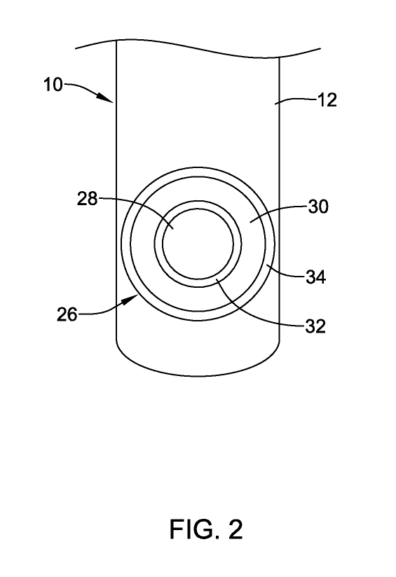

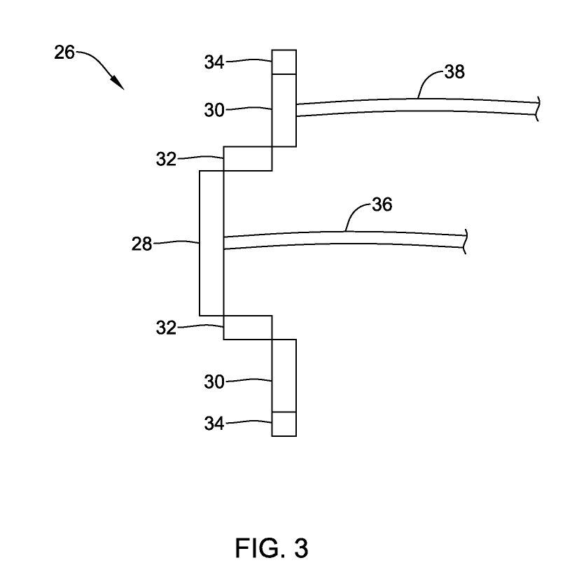

Some additional details regarding micro-electrode assembly 26 are shown in FIGS. 2-3. For example, in FIG. 2 it can be seen that micro-electrode assembly 26 may include a first or “inner” electrode 28 and a second or “outer” electrode 30. A layer of insulation 32 may be disposed between inner electrode 28 and outer electrode 30. In at least some embodiments, another layer of insulation 34 may be disposed along the perimeter of outer electrode 30. In embodiments where micro-electrode assembly 26 is disposed along distal ablation tip electrode 24, insulation 34 may insulate outer electrode 30 from distal ablation tip electrode 24.

The form of electrodes 28/30 may vary. In some embodiments, one or more of electrodes 28/30 may include an ablation electrode (e.g., an RF electrode, an ultrasound transducer, etc.). In some of these and in other embodiments, one or more of electrodes 28/30 may include a sensor. For example, one or more of electrodes 28/30 may include a voltage sensor, a temperature sensor, an ultrasound sensor, a force sensor, a contact sensor, a pressure sensor, an impedance sensor, an EGM sensor, or the like. In some embodiments, both of electrodes 28/30 may be the same type of sensor. In other embodiments, one of electrodes 28/30 may be one type of sensor (e.g., a voltage sensor) and the other of electrodes 28/30 may be another type of sensor (e.g., a temperature sensor, an ultrasound sensor, a force sensor, a contact sensor, a pressure sensor, an impedance sensor, an EGM sensor, or the like). In use, electrodes 28/30 (e.g., sensors 28/30) may be utilized to monitor the progress of a mapping and/or ablation procedure.

In some instances, electrodes 28/30 may be used in combination with distal ablation tip electrode 24 and/or ring electrodes 22. In other instances, the use of electrodes 28/30 may obviate the need for distal ablation tip electrode 24 and/or ring electrodes 22. Thus, one or more of distal ablation tip electrode 24 and/or ring electrodes 22 may be left off of system 10.

A first lead wire 36 may be coupled to inner electrode 28 as shown in FIG. 3. A second lead wire 38 may be coupled to outer electrode 30. Wires 36/38 may extend within shaft 12 to RF generator 14 and/or processor 16.

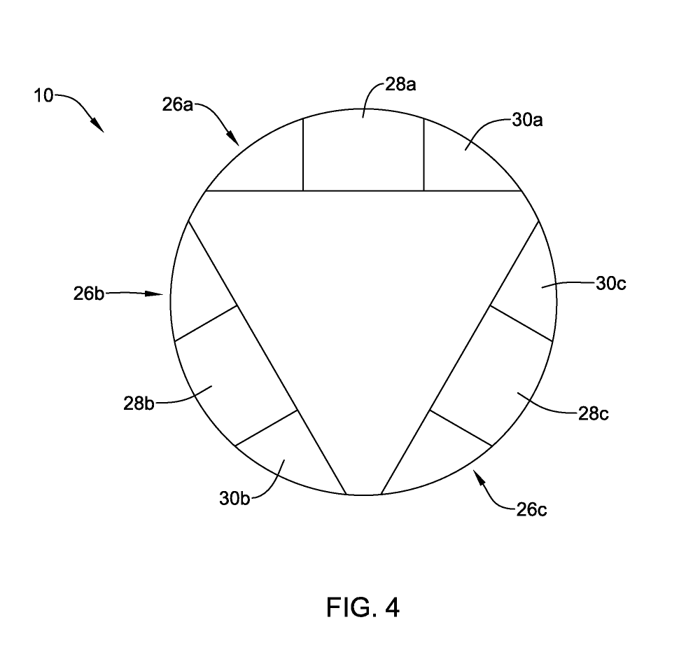

In at least some embodiments, a plurality of micro-electrode assemblies 26 may be included with system 10. For example, FIG. 4 illustrates that system 10 may include three micro-electrode assemblies 26a/26b/26c, each including inner electrode 28a/28b/28c and outer electrode 30a/30b/30c. The micro-electrode assemblies 26a/26b/26c may be evenly spaced about the circumference of shaft 12 (and/or distal ablation tip electrode 24 and/or system 10 in general). In other embodiments, micro-electrode assemblies 26a/26b/26c may be unevenly spaced about the circumference of shaft 12.

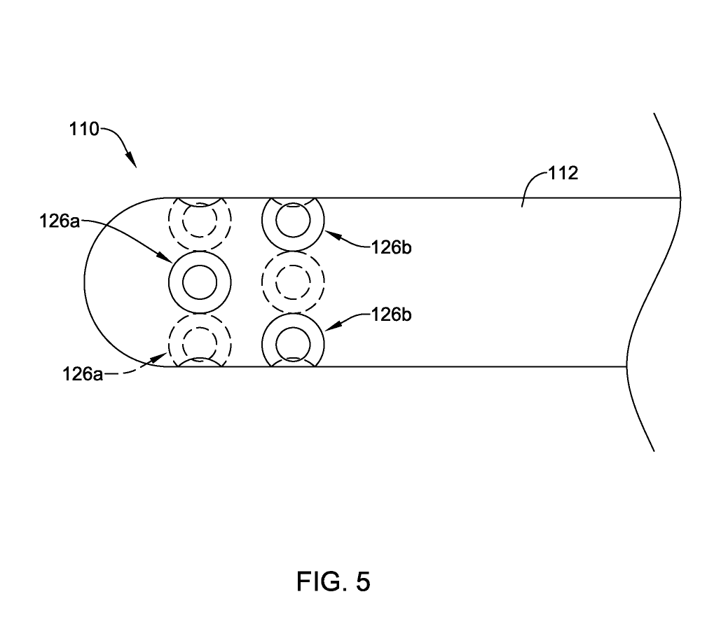

The number, arrangement, and configuration of micro-electrode assemblies 26 may vary. For example, FIG. 5 illustrates another example system 110, similar in form and function to other systems disclosed herein, that includes shaft 112 having a first row of micro-electrode assemblies 126a and a second row of micro-electrode assemblies 126b. In this embodiment, second row of micro-electrode assemblies 126b are offset or otherwise rotated (e.g., 45 degrees) relative to first row of micro-electrode assemblies 126a. Such an arrangement may allow for essentially 360 degrees of surface area coverage for the micro-electrode assemblies. While FIG. 5 shows two rows 126a/126b, any suitable number of rows may be utilized. Furthermore, while each row 126a/126b is shown to include three micro-electrode assemblies, evenly spaced apart, variations in the number of micro-electrode assemblies and the spacing thereof are also contemplated.

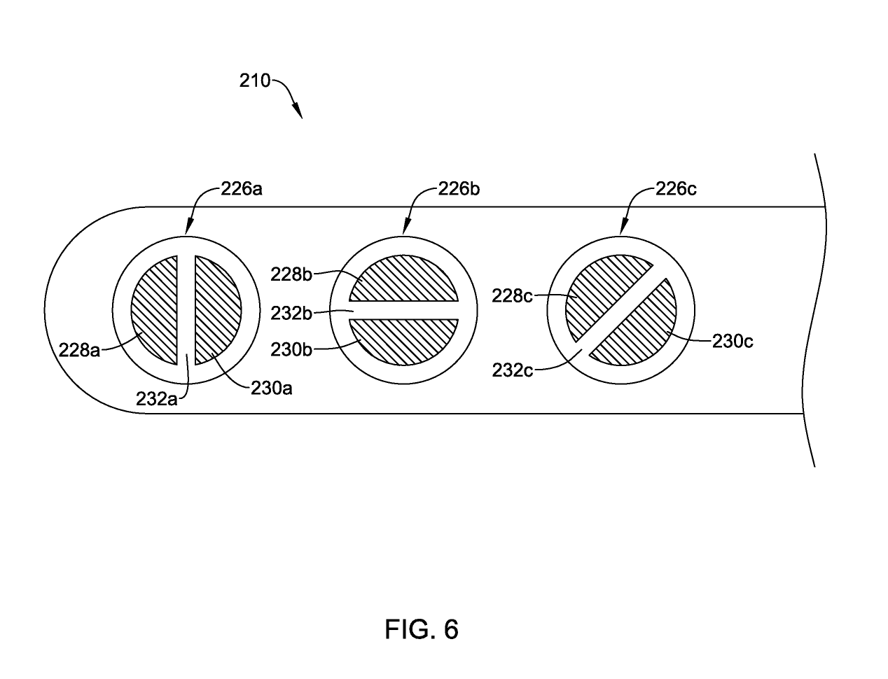

As suggested herein, a variety of shapes and arrangements are contemplated for the micro-electrode assemblies disclosed herein. For example, FIG. 6 illustrates a portion of another example system 210, which may be similar in form and function to other systems disclosed herein. System 210 may include a plurality of micro-electrode assemblies 226a/226b/226c. In this example, micro-electrode assemblies 226a/226b/226c include semi-circular electrodes oriented in different directions. For example, micro-electrode assembly 226a includes electrodes 228a/230a oriented in a direction that is transverse to the longitudinal axis of system 10. Insulating layer 232a may be disposed between electrodes 228a/230a. Micro-electrode assembly 226b includes electrodes 228b/230b oriented in a direction that is longitudinally aligned with the longitudinal axis of system 210. Insulating layer 232b may be disposed between electrodes 228b/230b. Micro-electrode assembly 226c includes electrodes 228c/230c oriented in a diagonal direction relative to the longitudinal axis of system 210. Insulating layer 232c may be disposed between electrodes 228c/230c. These are just examples. Variation in the number, shape, and arrangement of micro-electrode assemblies 226a/226b/226c and such variations may be utilized, where appropriate, with any of the systems disclosed herein.

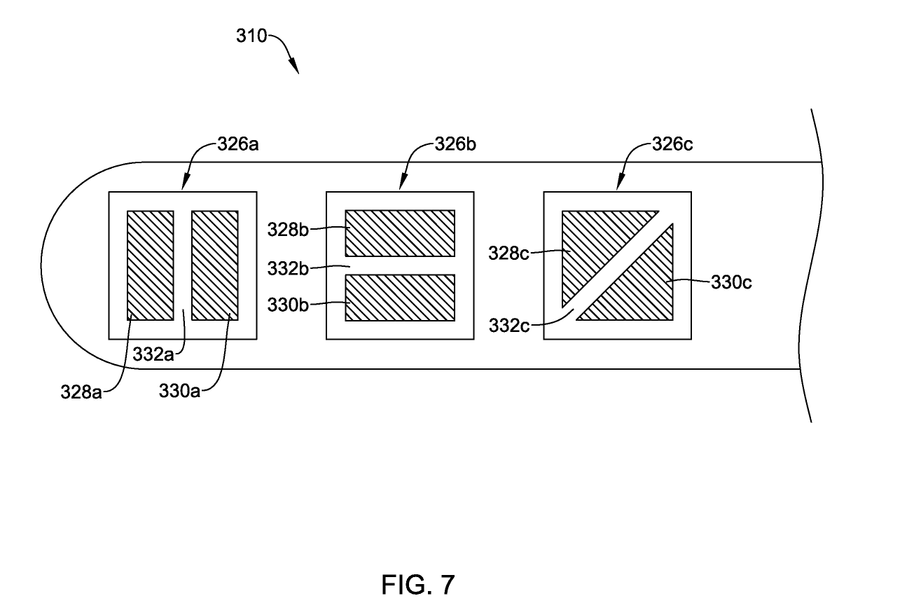

FIG. 7 illustrates a portion of another example system 310, which may be similar in form and function to other systems disclosed herein. System 310 may include a plurality of micro-electrode assemblies 326a/326b/326c. In this example, micro-electrode assemblies 326a/326b include rectangular electrodes oriented in different directions. For example, micro-electrode assembly 326a includes electrodes 328a/330a oriented in a direction that is transverse to the longitudinal axis of system 310. Insulating layer 332a may be disposed between electrodes 328a/330a. Micro-electrode assembly 326b includes electrodes 328b/330b oriented in a direction that is longitudinally aligned with the longitudinal axis system 310. Insulating layer 332b may be disposed between electrodes 328b/330b. Micro-electrode assembly 326c includes electrodes 328c/330c oriented in a diagonal direction relative to the longitudinal axis of system 310. In this embodiment, electrodes 328c/330c have a triangular shape. Insulating layer 332c may be disposed between electrodes 328c/330c. Variation in the number, shape, and arrangement of micro-electrode assemblies 326a/326b/326c and such variations may be utilized, where appropriate, with any of the systems disclosed herein.

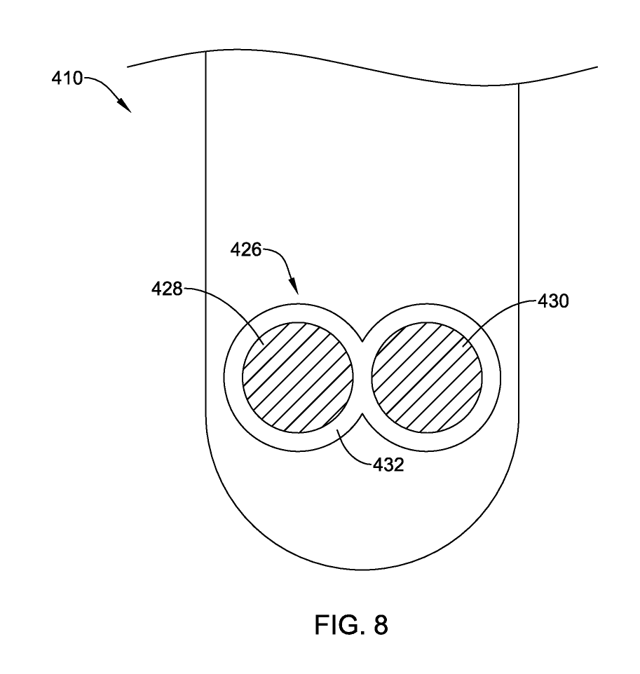

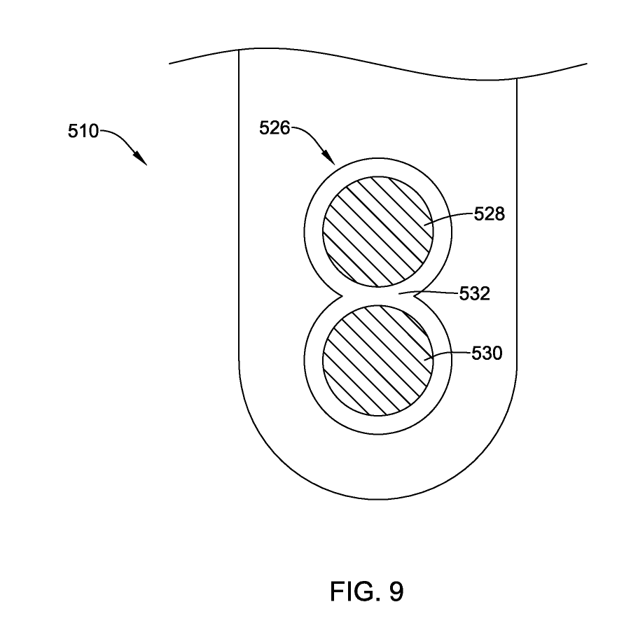

FIG. 8 illustrates a portion of another example system 410, which may be similar in form and function to other systems disclosed herein. System 410 may include micro-electrode assembly 426. In this example, micro-electrode assembly 426 includes two generally circular electrodes 428/430 arranged in a side-by-side configuration. Insulating layer 432 may be disposed around the periphery and/or between electrodes 428/430. Similarly, FIG. 9 illustrates a portion of another example system 510, which may be similar in form and function to other systems disclosed herein. System 510 may include micro-electrode assembly 526. In this example, micro-electrode assembly 526 includes two generally circular electrodes 528/530 arranged in an end-to-end configuration. Insulating layer 532 may be disposed around the periphery and/or between electrodes 528/530.

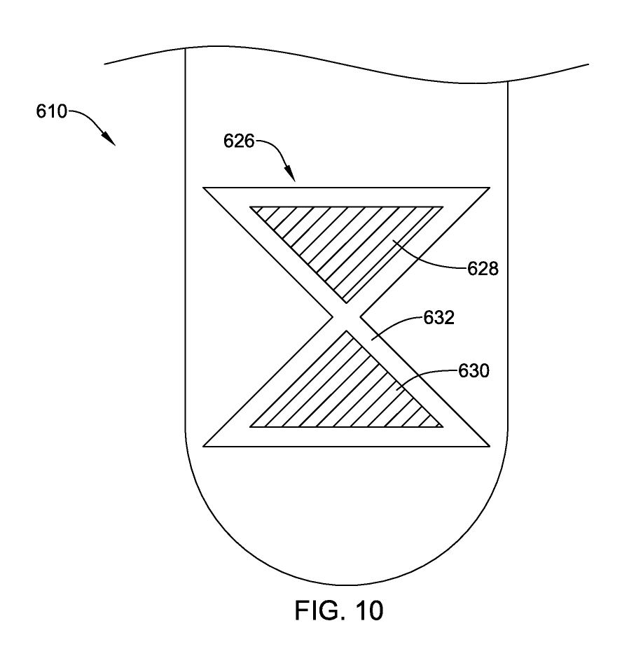

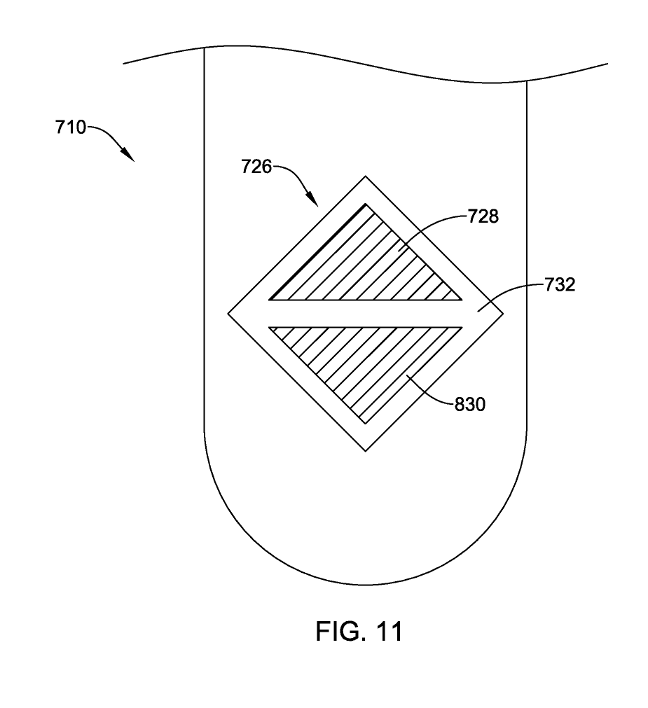

FIG. 10 illustrates a portion of another example system 610, which may be similar in form and function to other systems disclosed herein. System 610 may include micro-electrode assembly 626. In this example, micro-electrode assembly 626 includes two generally triangular electrodes 628/630 with a first arrangement (e.g., an apex of triangular electrodes 628/630 are posited next to each other). Insulating layer 632 may be disposed around the periphery and/or between electrodes 628/630. Similarly, FIG. 11 illustrates a portion of another example system 710, which may be similar in form and function to other systems disclosed herein. System 710 may include micro-electrode assembly 726. In this example, micro-electrode assembly 726 includes two generally triangular electrodes 728/730 with a varied arrangement (e.g., the hypotenuses of triangular electrodes 728/730 are posited next to each other). Insulating layer 732 may be disposed around the periphery and/or between electrodes 728/730.

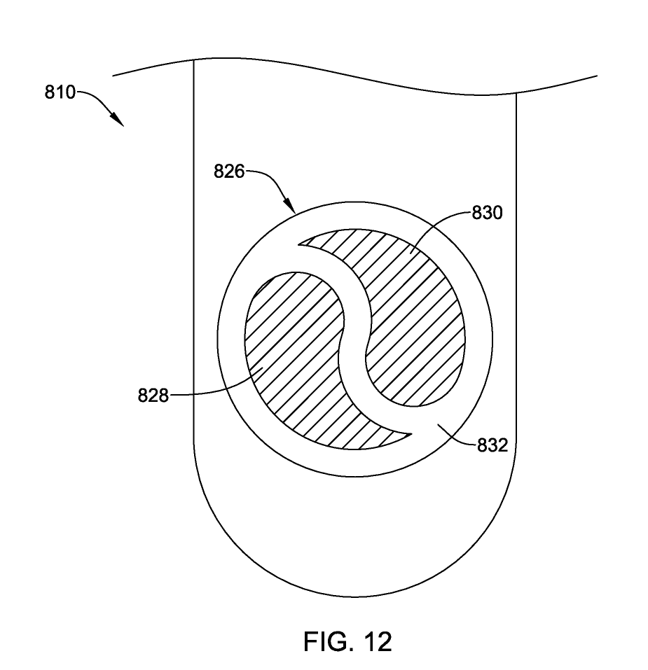

FIG. 12 illustrates a portion of another example system 810, which may be similar in form and function to other systems disclosed herein. System 810 may include micro-electrode assembly 826. In this example, micro-electrode assembly 826 includes two electrodes 828/830 having a rounded, tear-drop shape (e.g., a “ying-yang” shape). Insulating layer 832 may be disposed around the periphery and/or between electrodes 828/830.

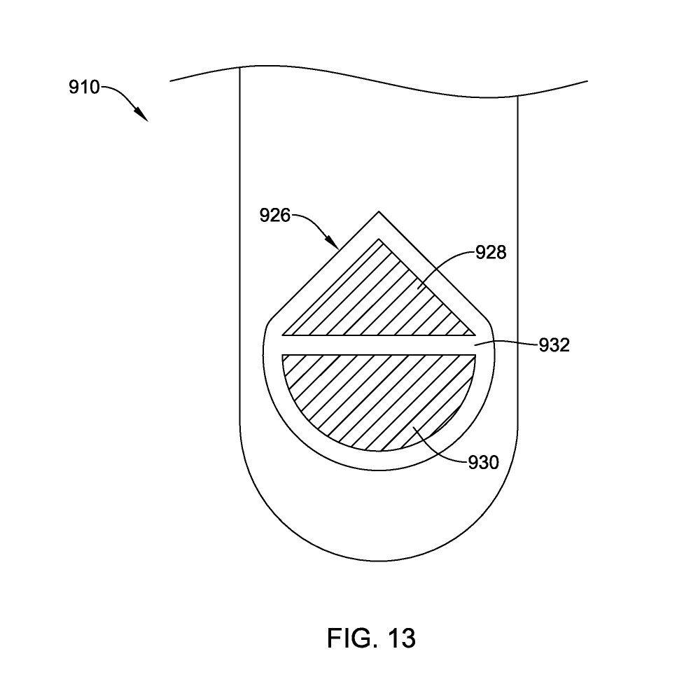

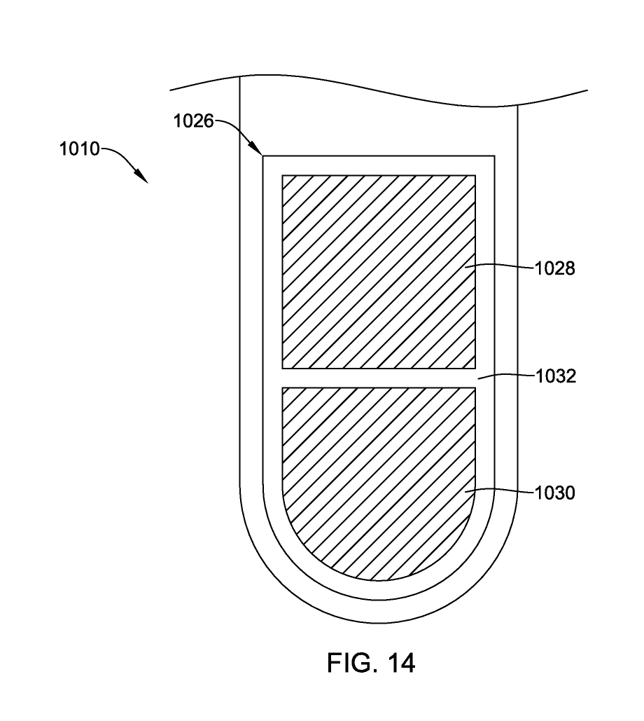

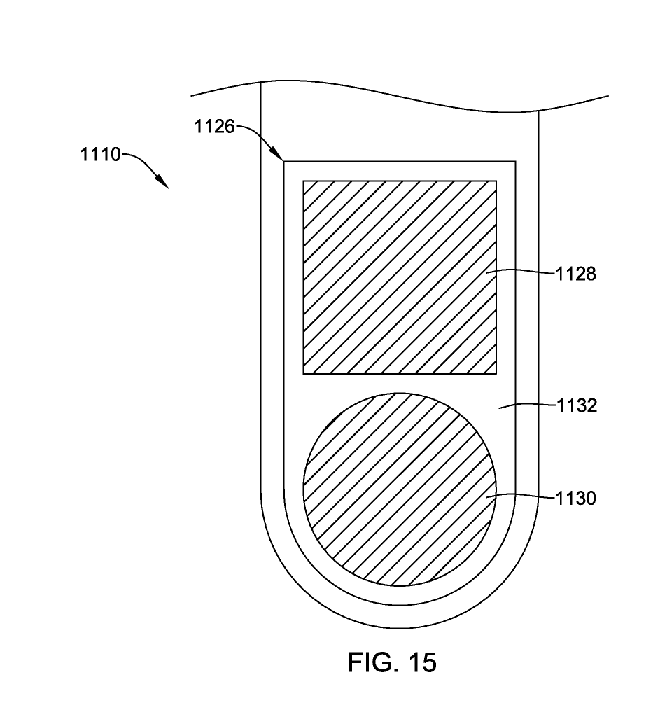

FIG. 13 illustrates a portion of another example system 910, which may be similar in form and function to other systems disclosed herein. System 910 may include micro-electrode assembly 926. In this example, micro-electrode assembly 926 includes two electrodes 928/930 having different shapes. For example, electrode 928 has a generally triangular shape and electrode 930 has a semi-circular shape. Insulating layer 932 may be disposed around the periphery and/or between electrodes 928/930. Similarly, FIG. 14 illustrates a portion of another example system 1010 including micro-electrode assembly 1026 with two electrodes 1028/1030 having different shapes. Insulating layer 1032 may be disposed around the periphery and/or between electrodes 1028/1030. Furthermore, FIG. 15 illustrates a portion of another example system 1110 including micro-electrode assembly 1126 with electrodes 1128/1130 having different shapes. Insulating layer 1132 may be disposed around the periphery and/or between electrodes 1128/1130. Collectively, these embodiments demonstrate that a variety of micro-electrode assemblies with differently shaped electrodes (including those shapes disclosed herein and other shapes) are contemplated.

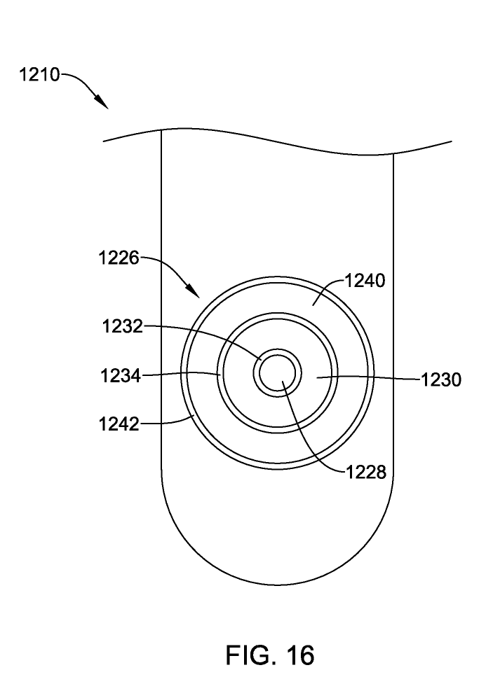

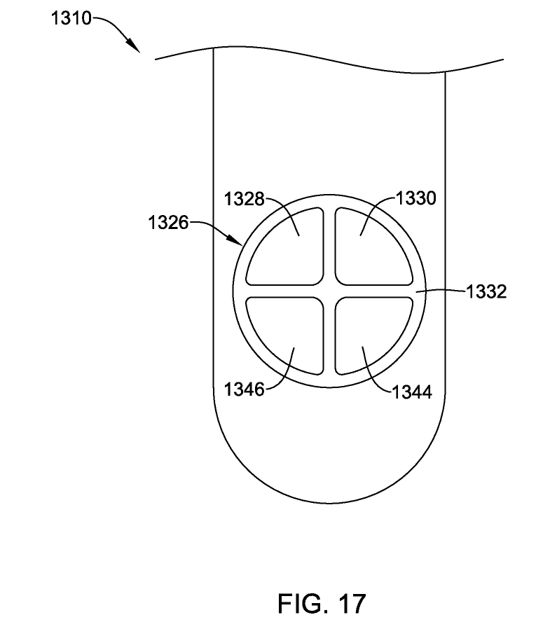

FIG. 16 illustrates a portion of another example system 1210, which may be similar in form and function to other systems disclosed herein. System 1210 may include micro-electrode assembly 1226. In this example, micro-electrode assembly 1226 includes three electrodes 1228/1230/1240. Insulating layers 1232/1234/1242 may be disposed around the periphery and/or between electrodes 1228/1230/1240. Other embodiments are contemplated that include more than three electrodes in a variety of different arrangements. For example, FIG. 17 illustrates a portion of another example system 1310, which may be similar in form and function to other systems disclosed herein. System 1310 may include micro-electrode assembly 1326. In this example, micro-electrode assembly 1326 includes four electrodes 1328/1330/1344/1346. Insulating layer 1332 may be disposed around the periphery and/or between electrodes 1328/1330/1344/1346.

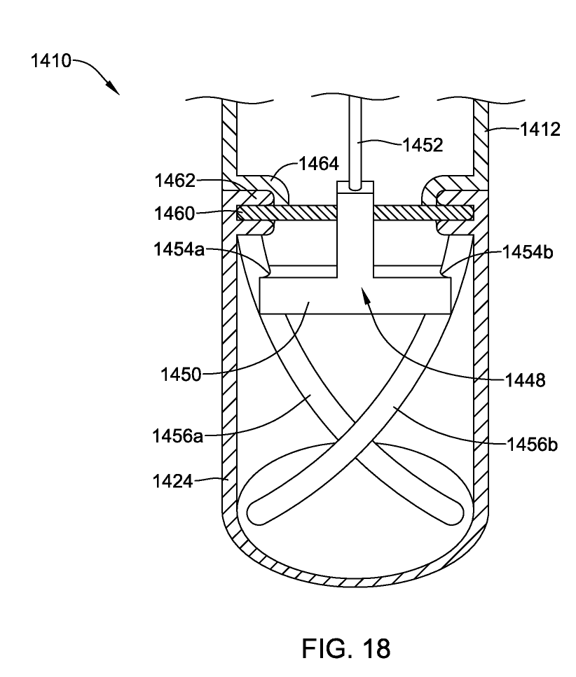

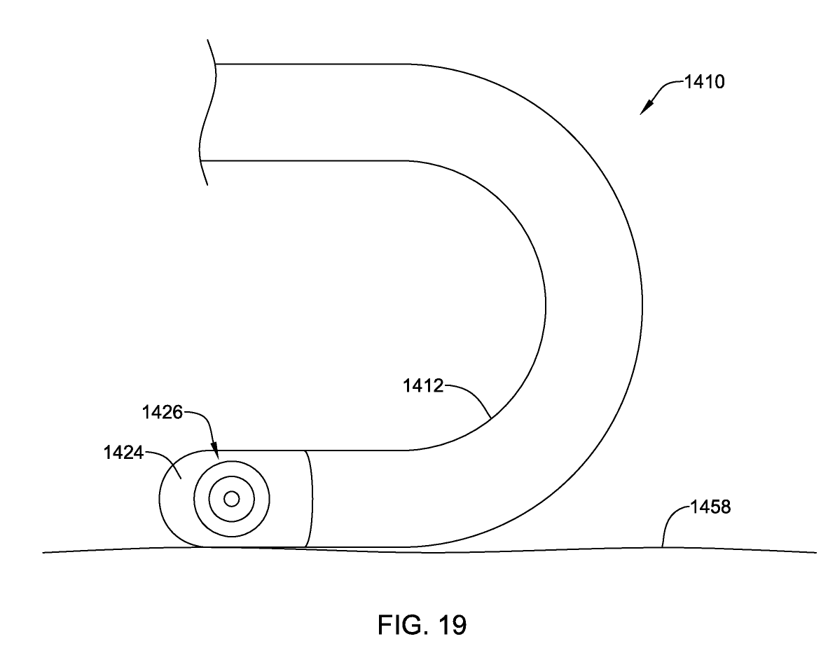

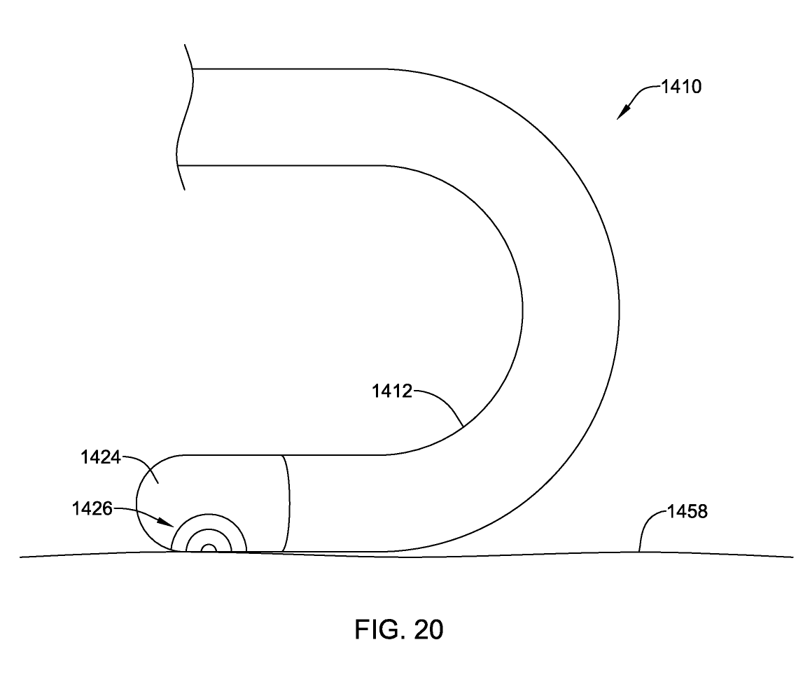

FIG. 18 illustrates a portion of another example system 1410, which may be similar in form and function to other systems disclosed herein. System 1410 may include micro-electrode assembly 1426 (not shown in FIG. 18, but can be seen in FIGS. 19-20) disposed along distal ablation tip electrode 1424. In some instances, it may be desirable for distal ablation tip electrode 1424 to be rotatable relative to shaft 1412. This may allow, for example, micro-electrode assembly 1426 to be rotated into a desired configuration so as to contact a target tissue.

In order to effect rotation, a number of different mechanisms may be utilized. For example, system 1410 may include a push-pull mechanism 1448. Push-pull mechanism 1448 may include a head region 1450 attached to a push-pull rod or wire 1452. Head region 1450 may have flanking keyed regions 1454a/1454b that are designed to be slidable along or otherwise follow rails/projections 1456a/1456b disposed along the interior of distal ablation tip electrode 1424. In addition, a rotatable member 1460 may be disposed within system 1410 that is rotatably coupled to a lip 1462 of distal ablation tip electrode 1424. In turn, a leg 1464 of shaft 1412 may be secured to rotatable member 1460. According to this arrangement, proximal or distal movement of rod 1452 may cause head region 1450 to move along rails 1456a/1456b and, thus, cause distal ablation tip electrode 1424 to rotate. For example, FIG. 18 illustrates system 1410 with distal ablation tip electrode 1424 in a first configuration where micro-electrode assembly 1426 is in a first configuration oriented generally away from a target tissue 1458. Proximal or distal movement of rod 1452 may cause distal ablation tip electrode 1424 to rotate such that micro-electrode assembly 1426 shifts to a second configuration oriented generally toward target tissue 1458.

While push-pull mechanism 1448 may be used to rotate distal ablation tip electrode 1424, this is just an example. A variety of rotatable mechanisms are contemplated. A suitable mechanism (such as push-pull mechanism 1448 and other contemplated mechanisms) may be used with system 1410 and/or other systems disclosed herein.

The use of a rotatable distal ablation tip electrode 1424 may be desirable for a number of reasons. For example, the use of a rotatable distal ablation tip electrode 1424 may allow for fewer micro-electrode assemblies 1426 to be used with system 1410 and/or other systems disclosed herein. This may include the use of a single micro-electrode assembly 1426 that can be rotated in a desired manner.

The materials that can be used for the various components of system 10 (and/or other systems disclosed herein) may include metals, metal alloys, polymers, metal-polymer composites, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, components of system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of system 10 to achieve the same result.

In some embodiments, a degree of Magnetic Resonance Imaging (MRI) compatibility is imparted into system 10. For example, components of system 10, or portions thereof, may be made of a material that does not substantially distort the image and create substantial artifacts (e.g., gaps in the image). Certain ferromagnetic materials, for example, may not be suitable because they may create artifacts in an MRI image. Components of system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A catheter for use in cardiac mapping and/or ablation, the catheter comprising:
an elongate catheter shaft having a distal ablation electrode region capable of ablating tissue;
a plurality of micro-electrode assemblies coupled to the distal ablation electrode region;
wherein at least one of the micro-electrode assemblies includes an inner electrode and an outer electrode disposed at least partially around the inner electrode; and
wherein at least one of the inner electrode and the outer electrode comprises a sensor.

2. The catheter of claim 1, wherein the distal ablation electrode region includes a platinum ablation tip electrode.

3. The catheter of claim 1, wherein the distal ablation electrode region is rotatable relative to the catheter shaft.

4. The catheter of claim 1, wherein three or more micro-electrode assemblies are disposed along the distal ablation electrode region.

5. The catheter of claim 1, wherein the micro-electrode assemblies are spaced substantially equidistant from one another about the circumference of the distal ablation electrode region.

6. The catheter of claim 1, wherein only one of the inner electrode and the outer electrode comprises a sensor.

7. The catheter of claim 1, wherein both the inner electrode and the outer electrode comprises a sensor.

8. The catheter of claim 1, wherein the sensor includes a voltage sensor.

9. The catheter of claim 1, wherein the sensor includes a temperature sensor.

10. The catheter of claim 1, wherein the sensor includes an ultrasound sensor.

11. The catheter of claim 1, wherein the sensor includes a force sensor.

12. The catheter of claim 1, wherein the sensor includes a pressure sensor.

13. The catheter of claim 1, wherein the sensor includes an impedance sensor.

14. The catheter of claim 1, wherein the sensor includes an EGM sensor.

15. A catheter for use in cardiac mapping and/or ablation, the catheter comprising:
an elongate catheter shaft having a distal ablation tip electrode;
a plurality of micro-electrode assemblies coupled to the distal ablation tip electrode;
wherein at least one of the micro-electrode assemblies includes a first sensor, a second sensor, and a layer of insulation disposed between the first sensor and the second sensor; and
wherein the first sensor, the second senor, or both includes a temperature sensor, an ultrasound sensor, a force sensor, a pressure sensor, an impedance sensor, or an EGM sensor.

16. The catheter of claim 15, wherein the first sensor and the second sensor are substantially congruent or geometrically similar.

17. The catheter of claim 15, wherein the first sensor and the second sensor have dissimilar shapes.

18. The catheter of claim 15, wherein the distal ablation tip electrode is rotatable relative to the catheter shaft.

19. The catheter of claim 15, wherein the distal ablation tip electrode includes three or more micro-electrode assemblies and wherein the micro-electrode assemblies are spaced substantially equidistant from one another about the circumference of the distal ablation tip electrode.

20. A method for mapping and/or ablation cardiac tissue, the method comprising:
advancing a mapping and/or ablation catheter through a blood vessel to a position within a cardiac chamber;
wherein the catheter comprises:
an elongate catheter shaft having a distal ablation electrode region capable of ablating tissue,
a plurality of micro-electrode assemblies coupled to the distal ablation electrode region,
wherein at least one of the micro-electrode assemblies includes an inner electrode and an outer electrode disposed at least partially around the inner electrode, and
wherein at least one of the inner electrode and the outer electrode comprises a sensor; and
activating the inner electrode, the outer electrode, or both.
